(19) United States
(12) Patent Application Publication
(10) Pub. No.: US 2006/0141483 A1
(43) Pub. Date: Jun. 29, 2006

(54) Stabilization of viral compositions

(76) Inventors: Gary J. Calton, Elkridge, MD (US); Rita Fishelevich, Baltimore, MD (US)

Correspondence Address: William S. Ramsey, 5253 Even Star Place, Columbia, MD, 21044, US

(21) Appl. No.: 11/021,289

(22) Filed: Dec. 23, 2004

(51) Int. Cl. C12Q 1/70; C12Q 1/68

(52) U.S. Cl. 435/6; 435/5

(57) ABSTRACT

This invention concerns stabilized virus compositions, preferably a herpesvirus which may be an attenuated or genetically modified herpes simplex virus or varicella zoster virus, and a method of stabilizing viruses and immunizing preparations by the addition of sugars, preferably glucose and amino acids, preferably lysine.

### CROSS-REFERENCE TO RELATED APPLICATIONS

Not Applicable.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### BACKGROUND OF THE INVENTION

(1) Field of the Invention

This invention relates to methods and compositions for stabilizing live virus and live virus compositions which are especially useful in immunizing preparations.

(2) Description of the Related Art including information disclosed under 37 CFR 1.97 and 37 CFR 1.98.

Live virus are usually unstable thermally and those of herpes virus are especially so. Herpes simplex virus is unstable even at −80° C. on extended storage. Vaccine preparations are often supplied as low-temperature frozen products or lyophilized products and numerous methods of stabilization have been evaluated.

U.S. Pat. No. 4,147,772 discloses the use of hydrolyzed gelatin and a polyhydric alcohol.

U.S. Pat. No. 4,337,242 discloses the use of L-glutamic acid and L-arginine in addition to hydrolyzed gelatin and a monosaccharide.

U.S. Pat. No. 4,500,512 discloses the use of phosphate buffer solution (PBS) containing calcium and magnesium ions, an amino acid and lactose or sorbitol for a Yellow Fever vaccine and gives examples of stabilization with very low concentrations of amino acids (0.005-0.05M).

U.S. Pat. No. 4,537,769 discloses the use of protein hydrolysates which are far superior to the amino acids glycine, isoleucine, leucine, lysine, histidine, methionine, phenylalanine, proline, serine, threonine, tryptophan, tyrosine, valine, alanine, aspartic acid and glutamic acid for influenza vaccines.

U.S. Pat. No. 4,985,244 discloses the stabilization of measles, mumps or rubella vaccines by lactose, saccharose, sorbitol, glutamate and gelatin hydrolysate.

U.S. Pat. No. 5,792,643 discloses the use of mannitol, lactose, sucrose and trehalose to stabilize a recombinant retrovirus.

U.S. Pat. No. 5,618,539 discloses the use of certain polyamines to stabilize polio virus, especially lysine at concentrations of 1-2 molar.

U.S. Pat. No. 5,948,411 discloses a live varicella virus vaccine stabilized by the addition of sucrose, lactose, sorbitol, cysteine, glutamate, gelatin and hydrolyzed gelatin from which the calcium and magnesium ions had been removed.

U.S. Pat. No. 6,258,362 discloses a lyophilized herpes virus composition stabilized by a polysaccharide having a molecular weight of 5,000 to 70,000 or a partially hydrolyzed peptone, a buffer and a monosaccharide.

Although various compounds have been used to stabilize live viral vaccines, it is clear that there exists a need for formulations which will improve the stability of viral preparations, especially those of the herpesvirus and more especially herpes simplex.

### BRIEF SUMMARY OF THE INVENTION

Embodiments of the present invention provide compositions for improving the stability of live virus vaccines containing live viruses such as herpes simplex viruses, especially types 1 and 2. Such improved stabilizers contain lysine at a concentration of 0.2 to 200 g/l and a sugar or sugar alcohol at 0.2 to 200 g/l. The inclusion of lysine and glucose in such compositions results in improved stability of the vaccines in both the liquid and solid state as well as improved yields during the process of harvesting virus for vaccine preparation. The use of lysine and glucose allows for the formulation of vaccine stabilizers that do not contain products of animal origin.

It is, accordingly, an object of embodiments of the present invention to provide an improved stability for a storage of live virus, especially herpes virus and more especially herpes simplex virus.

It is also the object of embodiments of the present invention to provide a process for stabilizing live virus, especially herpes virus and more especially herpes simplex virus.

Another object of embodiments of the present invention is to provide a stabilized formula for virus which may be used for preparation of immunizing preparations especially herpes virus and more especially herpes simplex virus preparations.

Yet another object of embodiments of the present invention is to provide a stabilized immunizing preparations especially herpes virus and more especially herpes simplex virus preparation which may be used in animals or humans.

Yet another object of embodiments of the present invention is to provide a method of stabilizing virus as it is extracted from cells or culture medium.

Yet another object of embodiments of the present invention is to provide a stabilized virus composition which does not contain added products of animal origin.

These and other objects of embodiments of the present invention will be apparent from the following description.

### BRIEF DESCRIPTION OF THE SEVERAL VIEWS OF THE DRAWING

Not Applicable.

### DETAILED DESCRIPTION OF THE INVENTION

Embodiments of the present invention are directed to compositions that are improved stabilizers for live virus vaccines. The improved stabilizers replace components of previous stabilizers which do not provide stability for frozen herpesvirus, especially herpes simplex and genetically modified herpes simplex. The present invention also provides immunizing compositions comprising virus with such improved stabilizers.

Embodiments of the present invention represent a significant improvement over the use of prior art stabilizers that included such components as serum albumin whether recombinant or animal in that lysine and glucose have been found to stabilize live viruses against inactivation without the necessity of adding protein, especially herpes viruses.

Embodiments of compositions according to the invention can generally be made in accordance with per-se known pharmaceutical practice so that they reach acceptable standards, e.g. of sterility.

The dose of virus in a frozen or lyophilized preparation according to an example of the invention can be chosen to be such as to yield, in the diluted or reconstituted liquid for injection, a dose of for example about 10³ to about 10⁸ pfu virus. A commonly chosen example of a volume of a dose for injection is about 0.1 to 0.5 ml.

The frozen or lyophilized preparation can be prepared from a liquid composition which is either of the same concentration in its principal components as the liquid to be reconstituted, or of greater or lesser concentration.

The moisture content of the lyophilized product can range from 0.5-15% and can be below about 10%, e.g. below about 5%, e.g. down to about 2% or less.

Also provided by embodiments of the invention is a process for producing a stabilized pharmaceutical preparation of a herpesvirus immunizing preparation, which is dispersible in aqueous liquid for injection, and which comprises lyophilizing a sterile aqueous composition containing (i) virus as active vaccine component, preferably a herpesvirus, e.g. an attenuated or genetically modified herpes simplex virus or varicella zoster virus, (ii) lysine at a final concentration in said sterile aqueous composition of 0.2 to 200 g/L (iii) and (iii) sugar, such as glucose or lactose, or a sugar alcohol, e.g. mannitol or sorbitol.

Additionally, a process of embodiments of the present invention for producing a stabilized pharmaceutical preparation of a herpesvirus immunizing preparation, which is a frozen stabilized preparation which comprises vialing a sterile aqueous composition containing (i) virus as active immunizing component, preferably a herpesvirus, e.g. an attenuated or genetically modified herpes simplex virus or varicella zoster virus, (ii) lysine at a final concentration in said sterile aqueous composition of 0.2 to 200 g/L (iii) and (iii) sugar, such as glucose or lactose, or a sugar alcohol, such as mannitol or sorbitol, which may then have water or a pharmaceutically suitable buffer added to dilute the preparation to a suitable level of osmolality such that it may be injected in a human or animal without harm.

The lyophilization of the product can be carried out over any suitable period according to conventional lyophilization practice, e.g. at a temperature below the glass transition temperature of the frozen liquid to be lyophilized, and the product can be in the form of a solid dried cake within a glass vial, preferably under sterile conditions. The freeze-drying process can comprise per-se known process steps to achieve two-stage drying in which a first stage of sublimation of the water content takes place at a temperature of for example about −40 deg. C. or lower, and then the temperature of the composition is raised to a higher temperature, e.g. 0 to +10 deg. C., when the drying has proceeded enough for the cake formed by the partially dried composition to retain its shape at the higher temperature, and a further amount of water is removed during and after such raising of temperature, still at reduced pressure.

The product can be rehydrated at convenience with sterile aqueous liquid, e.g. water for injection.

Also provided according to embodiments of the invention is a process for producing a liquid preparation of a virus vaccine for injection, which comprises dispersing or dissolving a sterile lyophilized preparation as specified above, e.g. a stabilized pharmaceutical preparation of a recombinant herpes simplex virus, in aqueous liquid for injection so as to produce a liquid composition of approximately isotonic concentration.

The compositions can also comprise other materials such as other colloids, which where present are preferably polysaccharides or polysaccharide derivatives such as hydroxyethyl starch. The virus of the formulations can generally comprise live virus, preferably attenuated or genetically modified.

The virus is preferably an infectious virus, e.g. a herpesvirus, and can be a genetically disabled virus of e.g. of one of the kinds described or referred to in WO 92/05263 (Immunology Ltd: Inglis et al); L H Nguyen, D Knipe et al, J Virol 66(12) (December 1992) 7067-7072; WO 94/01573 (Akzo: Peeters et al:) WO 94/03595 (Akzo: Visser et al:) WO 94/21807 (Cantab Pharmaceuticals Research Ltd: Inglis et al); WO 95/18852 (Harvard College and Dana-Farber Cancer Institute: D Knipe, et al); WO 96/04395 (Lynxvale Ltd: P Speck); WO 96/26267 (Cantab Pharmaceuticals Research Ltd: MEG Boursnell et al); U.S. Pat. No. 6,207,168 (University of Maryland at Baltimore: Aurelian); U.S. Pat. No. 6,054,131 (University of Maryland at Baltimore: Aurelian); and U.S. Pat. No. 6,013,265 (University of Maryland at Baltimore: Aurelian)

Embodiments of the invention are particularly applicable for example to herpesviruses and poxviruses among others. Particularly useful applications are for the stabilization of HSV, e.g. HSV-2, e.g. in the form of disabled HSV-2 such as that described in WO 94/21807 (Cantab Pharmaceuticals: Inglis et al), WO 96/26267 (Cantab Pharmaceuticals Research Ltd: MEG Boursnell et al), U.S. Pat. No. 6,207,168 (University of Maryland at Baltimore: Aurelian), U.S. Pat. No. 6,054,131 (University of Maryland at Baltimore: Aurelian), and U.S. Pat. No. 6,013,265 (University of Maryland at Baltimore: Aurelian), e.g. in embodiments wherein the virus carries exogenous genetic material encoding an immunomodulator or a heterologous antigen. Other herpesviruses such as for example varicella zoster virus, bovine herpes virus, and pseudorabies virus can also be formulated as described herein with similar results.

Examples of compositions of the invention can for example comprise immunogens and vaccines and viral vector preparations for in-vivo and ex-vivo use. The compositions can comprise immunogens other than the virus described above, e.g. immunomodulators such as interleukins, e.g. IL-12; and per-se known stabilizers and excipients such as may be desired for purposes of a given application in hand.

### EXAMPLE 1

An attenuated herpes virus preparation having a titer of 1×10⁸ pfu/mL had additional histidine, proline or histidine (0.002%) with glucose or proline (0.002%) with glucose added were maintained at 23° C., at 4° C. and at −20° C. At days 0, 7 and 21 the titers were determined by plaque assay. The following TABLE 1 gives the titers (000's pfu in 6 uL) found on the indicated day.

TABLE 1

| | Day 0 | | | Day 7 | | |
|---|---|---|---|---|---|---|
| | 23° | 4° | −20° | 23° | 4° | −20° |
| Histidine | 15 | 15 | 15 | 4 | 4 | 1 |
| proline | 15 | 15 | 15 | 7 | 3 | 20 |
| histidine + glucose | 15 | 15 | 15 | 8 | 6 | 30 |
| proline + glucose | 15 | 15 | 15 | 10 | 4 | 4 |
| control | 15 | 15 | 15 | 2 | 2 | 0 |

EXAMPLE 1 showed that each experimental treatment provided extended viability at each temperature compared to the controls.

### EXAMPLE 2

An attenuated herpes virus preparation having a titer of 1×10⁸ pfu/mL had additional lysine (0.9 M) or lysine (0.9 M) with glucose (2%) added and were maintained at room temperature, 23° C., and at 4° C. and at −20° C. At days 0, 14 and 28 the titers were determined by plaque assay. The following TABLE 2 gives the titers (000's pfu in 1 uL) found on the indicated day.

TABLE 2

| | Day 0 | | | Day 14 | | | Day 28 | | |
|---|---|---|---|---|---|---|---|---|---|
| | 23° | 4° | −20° | 23° | 4° | −20° | 23° | 4° | −20° |
| 0.9 M | 100 | 100 | 100 | 73 | 10 | 0 | 0 | 0 | 0 |
| lysine | | | | | | | | | |
| lysine + | 100 | 100 | 100 | 100 | 100 | 100 | 0 | 0 | 100 |
| glucose | | | | | | | | | |
| glucose | 100 | 100 | 100 | 0 | 0 | 0 | 0 | 0 | 0 |
| control | 100 | 100 | 100 | 0 | 0 | 0 | 0 | 0 | 0 |

EXAMPLE 2 showed an extension of virus lifetime by lysine or a mixture of lysine and glucose.

### EXAMPLE 3

An attenuated herpes virus preparation having a titer of 1×10⁷ pfu/mL in a final concentration of 0.875 M lysine and with 2% glucose added and were maintained at room temperature (approximately 23° C.), 4° C. and −20° C. At days 0 and 28 the titers were determined by plaque assay. The following TABLE 3 gives the titers (000's pfu in 7 uL) found on the indicated day.

TABLE 3

| | Day 0 | | | Day 28 | | |
|---|---|---|---|---|---|---|
| | 23° | 4° | −20° | 23° | 4° | −20° |
| 0.875 M lysine | 15 | 15 | 15 | 0 | 0 | 5 |

EXAMPLE 3 showed an extension of virus lifetime by lysine and glucose at −20° C.

### EXAMPLE 4

An attenuated herpes preparation having a titer of 1×10⁸ pfu/mL was diluted to a final concentration of 0.3, 0.5 and 0.7 M lysine with 2% glucose added and the aliquots were maintained at −20° C. At week 0 and 9, the titers were determined by plaque assay. The following TABLE 4 gives the titers (relative to day 0 at 100%) found at week 9.

TABLE 4

| | Week 0 | Week 3 |
|---|---|---|
| | −20° | −20° |
| 0.3 M lysine | 100 | 22 |
| 0.5 M lysine | 100 | 15 |
| 0.7 M lysine | 100 | 11 |

EXAMPLE 4 showed that all three concentrations of lysine with glucose extended the lifetime of herpes simplex at −20° C.

### EXAMPLE 5

Attenuated herpes preparations having titers of 6×10⁶ pfu/mL and 1.6×10⁶ pfu/mL in 0.3 M lysine with 2% glucose (final concentrations) were vialed and stored at −80° C. for 24 months. The titer was determined by plaque assay. The virus was stable for this period without a decrease in titer.

EXAMPLE 5 showed viability of herpes was maintained in 0.3 M lysine and 2% glucose for 24 months at −80° C.

### EXAMPLE 6

Attenuated herpes preparations, herpes simplex 2 in which the PK domain has been deleted, prepared by the methods disclosed in U.S. Pat. No. 6,207,168, incorporated herein by reference, having titers of 1.6×10⁶ pfu/mL were prepared with a stabilization formula of 0.3 M lysine and 2% glucose (final concentrations) and tested for therapeutic activity in guinea pigs. The guinea pigs were infected with HSV-2 (3×10⁶ pfu) in the footpad (day 0) and immunized with the preparation above or sham-immunized with PBS by subcutaneous inoculation in the flank on day 7 after and 17 after infection, and all animals were followed for the development of recurrent disease. The stabilized recombinant herpes simplex virus provided a 67% reduction in recurrences protection from recurrent disease in previously infected animals.

EXAMPLE 6 showed immunization with a herpes vaccine formulated with a stabilization formula of 0.3 M lysine and 2% glucose retained protective effect against herpes infection

### EXAMPLE 7

Attenuated herpes preparations having titers of 1.6×10⁶ pfu/mL were prepared with a stabilization formula of 0.3 M lysine and 2% glucose (final concentrations) and tested for therapeutic activity in humans who were known to be infected with genital herpes (HSV-2) and who had a minimum of 4-20 recurrences in the previous year. Prevention of recurrence was seen in 44% of the treated subjects whereas 87% of the placebo treated patients had a recurrence. Some of the treated patients received stabilized virus preparations as described in EXAMPLE 5 which had been prepared more than 12 months previously.

EXAMPLE 7 showed that herpes vaccine formulated with a stabilization formula of 0.3 M lysine and 2% glucose retained protective effect against herpes recurrence in humans known to be infected with genital herpes (HSV-2) after storage for 24 months.

It will be apparent to those skilled in the art that the examples and embodiments described herein are by way of illustration and not of limitation, and that other examples may be used without departing from the spirit and scope of the present invention, as set forth in the appended claims.

We claim:

1. A live virus preparation comprising:
(a) herpes simplex and
(b) lysine at a concentration of 0.2 to 200 g/l. and
(c) a sugar or sugar alcohol at 0.2 to 200 g/l.

2. The preparation of claim 1 wherein the sugar or sugar alcohol is glucose.

3. A live virus preparation prepared by lyophilizing the preparation of claim 1.

4. A process for preparing stabilized vaccines containing attenuated live viruses, said process comprising contacting attenuated live viruses with an effective amount of the stabilizing agent of claim 1.

5. The process of claim 4 wherein said live virus is a herpes virus.

6. The process of claim 4 wherein said herpes virus is a herpes simplex type 2.

7. The process of claim 6 wherein said herpes virus is a herpes simplex type 2 wherein the PK domain has been deleted.

8. The process of claim 4, wherein said contacting is conducted prior to a lyophilization step.

9. A stabilizing agent for live herpes virus viral immunizing agent comprising lysine and glucose.

10. The stabilizing agent of claim 9 wherein said immunizing agent is a herpes virus type 2.

11. The stabilizing agent of claim 10 wherein said herpes virus is a herpes simplex type 2 in which the PK domain has been deleted.

12. A method of preparing a live virus immunizing agent that comprises mixing a live herpes virus with a stabilizer comprising an aqueous solution of a sugar or sugar alcohol at 1-100 g/l and lysine at 2 to 20 g/l.

13. The method of claim 1 wherein said live virus is a herpes simplex type 2 virus in which the PK domain has been deleted.

14. A method of harvesting live herpes virus for use in a live virus immunizing agent that includes the step of disrupting cells containing said virus in the presence of a stabilizer comprising an aqueous solution of a sugar or sugar alcohol at 1-200 g/l and lysine at 0.2 to 200 g/l.

15. The method of claim 14 wherein said sugar or sugar alcohol is glucose.

16. The method of claim 14 wherein said live herpes virus is a herpes simplex type 2 virus.

17. The method of claim 16 wherein said herpes virus is a herpes simplex type 2 in which the PK domain has been deleted.

18. A method of harvesting herpes simplex virus for use in a live virus immunizing agent that includes the step of separating the growth medium from the cells in which the herpes simplex is grown and adding a stabilizer to the growth medium containing excreted virus comprising lysine and sugar or sugar alcohol to provide a final concentration of the sugar or sugar alcohol at 1-200 g/l and of the lysine at 0.2 to 200 g/l.

19. The method of claim 18 wherein said sugar or sugar alcohol is glucose.

20. The method of claim 18 wherein said herpes virus is herpes simplex type 2.

21. The method of claim 20 wherein said herpes virus is a herpes simplex type 2 in which the PK domain has been deleted.
